(19) United States
(12) Patent Application Publication
(10) Pub. No.: US 2021/0121516 A1
(43) Pub. Date: Apr. 29, 2021

(54) Natural asthma treatment

(71) Applicant: Hope Christine Radellant, New York, NY (US)

(72) Inventor: Hope Christine Radellant, New York, NY (US)

(21) Appl. No.: 16/667,450

(22) Filed: Oct. 29, 2019

(51) Int. Cl. A61K 36/906; A61K 36/73; A61K 36/752; A61K 35/644; A61K 36/8962

(52) U.S. Cl. CPC A61K 36/906; A61K 36/73; A61K 36/8962; A61K 35/644; A61K 36/752

(57) ABSTRACT

The present invention is directed to an all natural treatment for asthma. The treatment is in liquid form and uses no toxic chemicals or preservatives. The composition includes ginger juice, apple cider vinegar, lemon, honey, CBD oil, and garlic.

### BACKGROUND OF THE INVENTION

### 1. Field of the Invention

The present invention relates to treatments for asthma. More specifically, the invention relates to an all natural treatment for asthma which uses no toxic chemicals or preservatives.

### 2. Description of the Prior Art

Atmospheric pollution has become remarkably increased due to exhaust gases, chemical substances or dusts from automobiles or factories, and accompanying this, the number of patients with bronchial asthma has been increased.

Bronchial asthma is a severe disease in which constriction and contraction of the bronchial smooth muscle occur at the time of the seizure, and at the time of status asthmatics, a patient is lead to suffocation death from the state of very painful dyspnea. Bronchial asthma is the most frequent cause for hospitalization not only in adults but also in children and, by present medical care, bronchial asthma is thought to be a disease for which prevention and treatment corresponding to asthma in individual patients are difficult.

Currently, for treating such chronic bronchial asthma, adrecocortical hormones, a so-called steroidal agent is mainly used. However, this drug is effective in the treatment, but it causes problematic side effects. For example, glucocorticosteroids which are known as an effective anti-asthma drug actually has only transient abating effect to the symptom and, as a sacrifice, the well-known side effects such as osteoporosis, obesity, hypertension and diabetes are accompanied. Other pharmaceutical treatments have similar or worse side effects. Accordingly, it is desirable to have an asthma treatment which is formulated from all natural ingredients, all of which are certified organic.

The present invention is directed to an all natural treatment for asthma. The treatment is in liquid form and uses no toxic chemicals or preservatives. The composition includes ginger juice, apple cider vinegar, lemon, honey, and garlic.

### SUMMARY OF THE INVENTION

It is a major object of the invention to provide an all natural remedy for asthma.

It is another object of the invention to provide an all natural remedy for asthma which uses all organic ingredients.

It is another object of the invention to provide an all natural remedy for asthma which uses no preservatives.

It is another object of the invention to provide an all natural remedy for asthma which is in liquid form.

It is another object of the invention to provide an all natural remedy for asthma which contains CBD oil.

### DETAILED DESCRIPTION OF THE PREFERRED EMBODIMENTS

The present invention is directed to an all natural composition for the treatment of asthma. The dosage of the inventive composition may vary depending on the patient's weight, age, sex, physical condition and diet, the time of administration, the mode of administration, excretion rate, and the severity of the disease. The inventive composition may be administered once or several times a day at predetermined time intervals according to the physician's or pharmacist's judgment, or in the event of an asthma attack.

In a preferred embodiment, the composition is in liquid form. The ingredients are ginger juice, apple cider vinegar, lemon, honey, CBD oil, and garlic. The ingredients are added in equal volume by weight. The composition is prepared by mixing the ingredients at room temperature in a suitable vessel until all of the components are in solution, which is visually determined.

There is no particular limitation on liquid components, and the composition may further contain various flavoring agents like conventional beverages. In addition, examples of flavoring agents that may be used in the present invention include natural flavoring agents (taumatin, stevia extracts such as levaudioside A, glycyrrhizin, etc.).

It is to be understood that the present invention is not limited to the sole embodiment described above, but encompasses any and all embodiments within the scope of the following claims:

I claim:

1. A composition for treating asthma consisting of ginger juice, apple cider vinegar, lemon, honey, CBD oil, and garlic, in equal parts by weight.
